(19) United States
(12) Patent Application Publication
(10) Pub. No.: US 2014/0327460 A1
(43) Pub. Date: Nov. 6, 2014

(54) Test Apparatus and Method for Determining Long Term Reliability of an Implantable Device

(71) Applicant: Second Sight Medical Products, Inc., San Fernando, CA (US)

(72) Inventors: David Daomin Zhou, Saugus, CA (US); Alexander Istomin, Newhall, CA (US); James S. Little, Newhall, CA (US); Robert J. Greenberg, Los Angeles, CA (US)

(21) Appl. No.: 14/270,154

(22) Filed: May 5, 2014

Related U.S. Application Data
(60) Provisional application No. 61/819,464, filed on May 3, 2013.

(51) Int. Cl. A61N 1/372; A61N 1/36

(52) U.S. Cl. CPC A61N 1/372; A61N 1/36046
USPC 324/750.03

(57) ABSTRACT

The present invention is a system and method for testing the long term reliability of an implantable device. The system provides a vessel containing temperature controlled buffered saline. A support structure suspends a test device in the vessel so it is submerged in the buffered saline and provides mechanical stress on the implantable devices. The test device is eclectically connected to a programmable signal generator and sensors to actively determine the integrity of the device during active testing.

### FIELD OF THE INVENTION

The present invention relates to test apparatuses and methods, and more particularly to system to more accurately represent the conditions within human body.

### BACKGROUND OF THE INVENTION

Retinal prostheses and other active implantable medical devices are susceptible to damage by body fluids over time. Long-term reliability of retinal prostheses requires hermetic packaging to protect the electronic circuitry of the implant from the harsh environment of the human body and a robust high-density electrode array for safe chronic stimulation. In addition to lifetime testing under normal use conditions, accelerated lifetime testing has been widely used to predict the implants' life and to better understand their failure modes. FDA guidance recommends designing implanted components of a retinal prosthesis to withstand a minimum of 5 years simulated use.

### SUMMARY OF THE INVENTION

The present invention is a system and method for testing the long term reliability of an implantable device. The system provides a vessel containing temperature controlled phosphate buffered saline (PBS). A support structure suspends a test device in the vessel so it is submerged in the PBS and provides mechanical stress on the implantable devices. The test device is eclectically connected to a programmable signal generator and sensors to actively determine the integrity of the device during active testing.

### BRIEF DESCRIPTION OF THE DRAWINGS

FIG. 1 is a block diagram showing the dynamic implant lifetime test system.

FIG. 2 is a view of complete test setup.

FIG. 3 is a perspective view of a three axis accelerometer for controlled movement of the test device.

FIG. 4 is a perspective view of a test device suspension system including support of a primary coil.

FIG. 5 is a perspective view of the test device in the suspension system.

### DETAILED DESCRIPTION OF THE PREFERRED EMBODIMENT

It should be noted that the preferred embodiment is described in terms of a system for testing the implantable portion of a visual prosthesis. It should be clear that the system is adaptable to other active implantable devices. Discussion of testing the movement of an eye can be applied to other muscle movement as it applies to other implantable devices.

### Methods

The preferred visual prosthesis implant is an epiretinal prosthesis that includes a receiver antenna, electronics package, and an electrode array. The array has 60 platinum based electrodes arranged in a 6×10 grid. The flexible polymer thin-film electrode array, which follows the curvature of the retina, is attached to the retina over the macula with a retinal tack. Lifetime testing of the preferred visual prosthesis implant has been conducted at the component, subsystem and final device levels. Long-term stability of the implants is assessed in vitro through active soak tests under constant pulse stimulation. The packages are tested in PBS solutions at body temperature, or elevated temperatures for accelerated tests.

In the final device tests, the implants are attached to the silicone eye model to simulate the actual implanted condition. During some soak tests, a motor moves the entire eye model to simulate micro motion of a human eye. The device functionality, visual appearance, and material changes are monitored through the course of the lifetime test.

Once implanted, the retinal prosthesis located on the inside and outside of the eye will experience stresses from both the harsh environment of the human body and saccadic movement. A reasonable test duration is 5 years, however if implants fail before five years, patients would be required to undergo surgery that could have been otherwise prevented. By utilizing the implant Dynamic life test system (IDLT), engineers will determine how to improve the reliability and functionality of the retinal prosthesis. In attempting to mimic the conditions that a real implant will experience, a system must meet these key design objectives:

Reliability—the testing apparatus must complete multiple tests with little maintenance, if the testing apparatus fails before the implant, the goal of testing an implant from start to finish is unobtainable.

Accurate saccadic movement—by accurately creating saccadic-like movements, the test accurately mimics the forces and/or movements that the implant would experience.

Accelerated time—the testing apparatus must simulate an accelerated life test.

Increased heat—the test must maintain a steady temperature (above body temperature) to decrease the test time.

Visual inspection—a lab technician or any person must be able to look at the implant during the test and determine if it has failed.

Ability to test individual implants—implants are tested in individual PBS baths. This allows more flexibility in testing including variable speeds, temperatures, and other conditions.

Periodic data recording—ensures that the implant is working properly and provides test data for engineering analysis.

Data recording during failure—if the implant fails, all relevant data must be recorded to determine the source of failure.

The user inputs will be contained in a system configuration file. The inputs are as follows: Sensor polling frequency (Hz); desired temperature (° C.); desired angular displacement) (° ; desired motor frequency (Hz).

The system outputs to the user will be contained in space-limited files. The outputs are as follows: video processing unit (VPU) statistics (current status of electrode array); Desired angular acceleration (°/s²); Actual angular acceleration obtained from accelerometer (°/s²); Desired motor frequency (Hz) Actual motor frequency (Hz) Desired heat pump temperature (° C.); Actual heat pump temperature obtained from external probe (° C.).

Reliability plays the most critical role in the final design. To start, the testing apparatus must function throughout the life of a test which can range from zero to five years or possibly more. Additionally, the testing apparatus should be reusable through multiple tests with limited maintenance time.

The system must provide the ability to reuse testing apparatus through multiple tests of minimum length 5 years with t minutes required maintenance time per week.

The design must incorporate an accelerated life test which is accomplished two ways, elevated temperature and elevated frequency of eye oscillation. The system must provide the ability to adjust, maintain and monitor temperature between 37° C. and 87° C.

The system must mimic eye movement. Another key design objective includes the ability to mimic saccadic eye movement that the retinal prosthesis will experience after implantation. The system must provide the ability to adjust, maintain and monitor eye movement with an angular displacement between 0° and +/−30° and a frequency of oscillation between 1 Hz and 10 Hz.

The system must mimic the harsh environment of the eye. The retinal prosthesis will be subject to the harsh environment of the eye. The retinal prosthesis must be completely submerged in PBS throughout the duration of the test.

The tester needs to be able to visually inspect the integrity of the implant at all times. The vessel containing the implant must be transparent, and the implant must be visible to the naked eye at all times of the test.

The system must provide the ability to test implants independently of each other. The testing apparatus must test a single implant at a time.

The system must provide maintain VPU communication—The VPU is in constant communication with the implant through means of RF communication. This RF communication cannot be interrupted. The system must maintain communication with the VPU at all times by maintaining a distance of 10 mm to 20 mm between RF communication coils. The test apparatus cannot allow for no metal materials located neither between RF communication coils nor inside the silicone eye model. The system must provide the ability to stimulate specific electrodes on the electrode array contained in the implant. Additionally, the tester needs to know whether not the electrode was successfully stimulated. The system must provide bidirectional communication with the VPU in order to stimulate electrodes in the electrode array and the resulting data.

The system must provide for mechanisms to gather test data over a long period of time that can be used to make engineering decisions regarding the retinal prosthesis. The system must record relevant test data every t seconds.

The system must produce a testing environment that matches the actual environment that the human body and provide for a retinal prosthesis implant in that environment. This environment will be mimicked by utilizing a temperature elevated PBS solution and angular rotation, which mimics the saccadic eye movement.

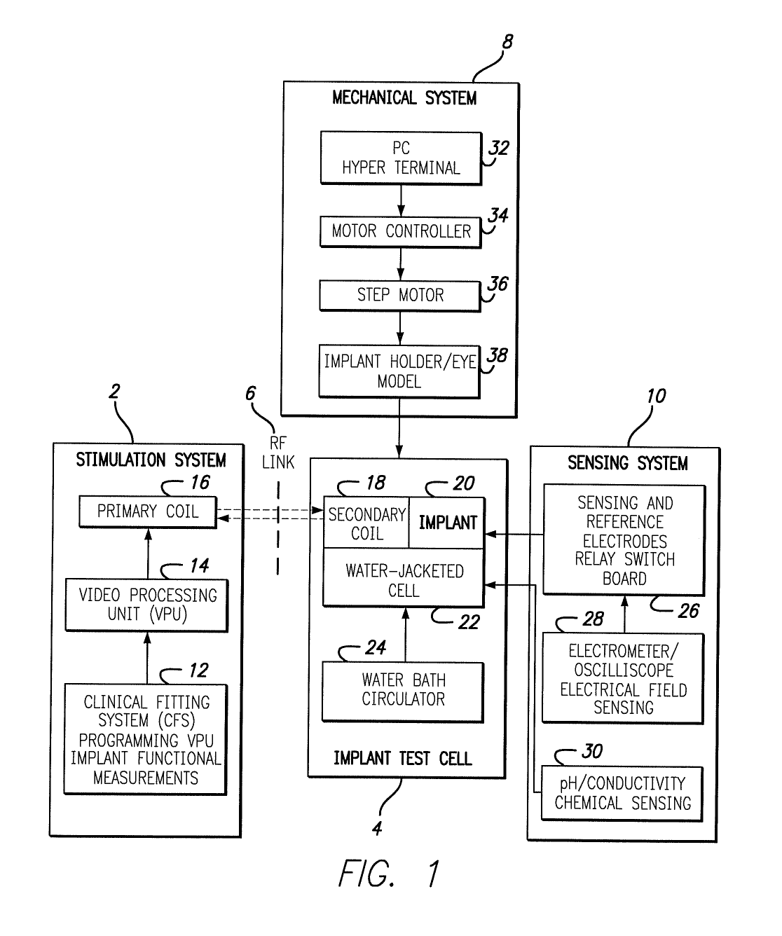

The user interface is designed to be a robust, stream-lined algorithm that provides the user with a simple command line interface to configure the file that determines the user inputs to the IDLT. Referring to FIG. 1, the functional structure of the test system has four main components each including subcomponents. The stimulation system 2 mimics the external components of a visual prosthesis and provides stimulations signals to the implant test cell 4 through an RF link 6, as stimulation signals would be provided in an actual implanted visual prosthesis. The mechanical system 8 provides motion to the test sample mimicking the saccades of an eye. The sensing system 10 records the performance of the test sample. The stimulation system 2 includes the normal components of the external part of a visual prosthesis. The clinical fitting system 12 is a computer where a clinician would provide adjustments to a visual prosthesis. In this case, the clinical fitting system 12 is programmed to control the testing regime. The clinical fitting system 12 controls a video processing unit 14. This is the same as the video processing system that would be worn by the patient under normal use. The video processing unit 14 drives the primary coil 16. The primary coil 16 sends signals over the RF link 6 to the secondary coil 18 inside the test cell 5. The secondary coil 18 forms part the implant 20, which is the test sample. The implant 20 is within a water jacketed cell 22 that is provided with PBS solution at the correct test temperature by the water bath circulator 24. The results of the tests are determined by the sensing system 10. A reference electrode and a sensing electrode within the water jacket cell 22 is connected to a relay switch board 26 which connects the sensing electrode to an electrometer or oscilloscope 28 for electrical field measurements. A chemical sensing electrode, such as a pH electrode, ion-selective electrode or conductivity electrode, within the water-jacketed cell 22, is connected to the chemical sensing system 30. The implant 20 is suspended in the water-jacketed cell 22 by the mechanical system 8. The mechanical system includes a hyper terminal 32 for control of the mechanical system. This can be the clinical fitting system 12 or a separate computer. The motor controller 34 controls the step motor 36 which moves the implant holder 38. The implant 20 is within the implant holder 38 and is moved according to the motor controller.

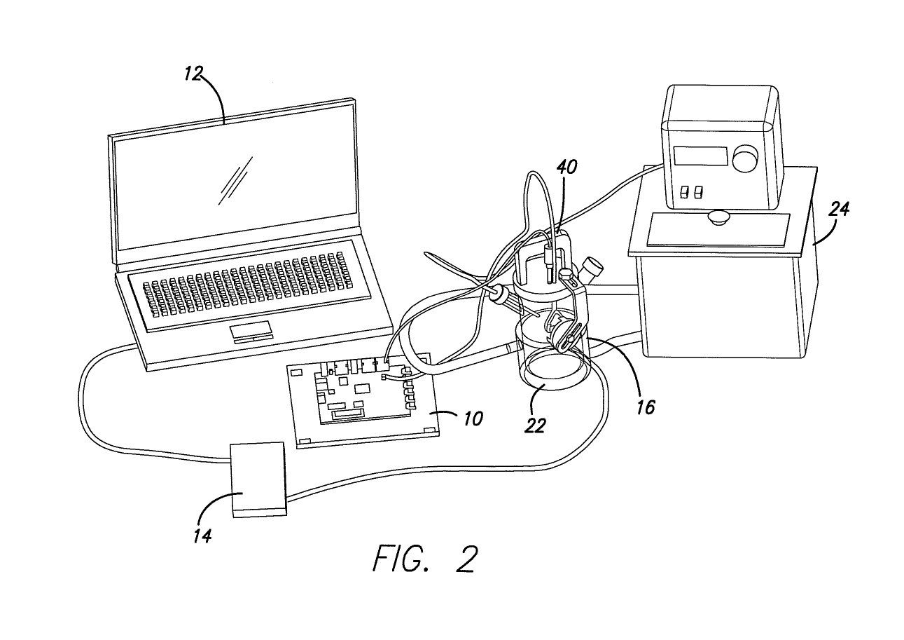

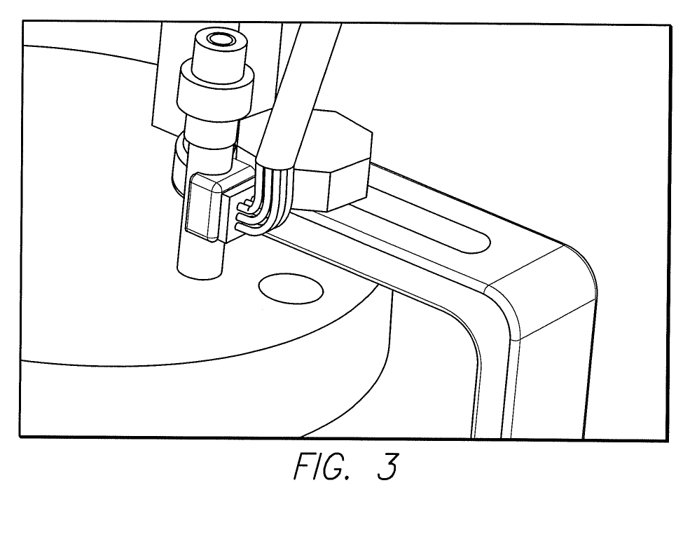

FIG. 2 is a physical view of complete test setup shown functionally in FIG. 1. The system can be broken into three major components. Each subcomponent is shown in the FIGS. 3-5. The CFS 12 is run on a laptop computer when programs the video processing unit 14. The video processing unit 14 drives the primary coil 16, which is held next to the water-jacketed cell 22 to maintain the same relationship with the secondary coil 18 as if were implanted in a person.

Referring to FIG. 3 is a perspective view of a three axis accelerometer for controlled movement of the test device. The three axis accelerometer is capable of measuring the step motor's 36 mechanical movements such as angular acceleration and angular displacement. The stepper motor 36, can only rotate the device on one axis. The accelerometer pad consists of two pieces that are removable and can be adhered to the shaft during installation. Since the shaft must fit through the bearing in the water jacketed cell 22 lid, the two halves of the accelerometer pad are glued onto the shaft after insertion though the water jacketed cell 22 lid. The accelerometer pad has a raised platform to allow space for the soldered wires to emerge.

The DC voltage servomotor is a precise position control motor. Due to their electronic commutation, the service life of these motors is only limited by the bearings. The brushless DC motor can achieve a continuous output torque up to 22 mNm at speeds up to 49,000 rpm. The Brushless DC motor comes in a sterilized version capable of withstanding an autoclave environment. The housing of the motor is black to facilitate optimum heat dissipation. This specific brushless DC motor will be combined with an encoder for a complete miniature DC drive system. The 2 channel magnetic encoders allow for precise control of speed and direction of rotation as well as the positioning of the drive shaft. This series of motor can also be combined with a gearbox. The optional planetary and spur gearboxes are available in a large range of sizes with various reduction ratios allowing an output torque up to 700 mNm.

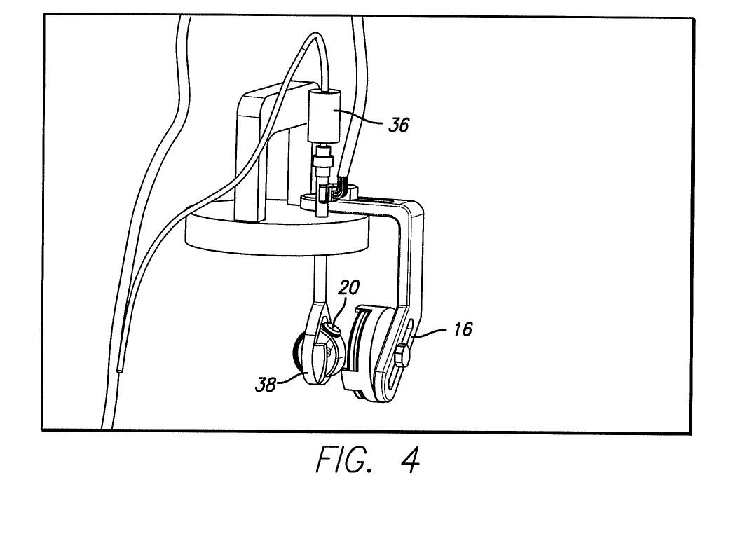

Referring to FIG. 4. Is a perspective view of a test device suspension system 40, including support of a primary coil 16. The test device suspension system 40 holds the step motor 36, which holds and moves the implant holder 38 within the water jacketed cell 22. The suspension fixture implant holder 38 has two fins that hold the silicone eye even more snugly in the fixture. The two fins are angled so that the user can easily insert and remove the silicone eye. The groove on the top and bottom of the fixture are for the sclera strap. By not having a flange on the silicone eye model, it allows the sclera strap to sit directly on the silicone eye, more closely simulating implant conditions. The gap at the top of the cylinder has enough clearance between the package and the fixture to ensure they do not touch during testing.

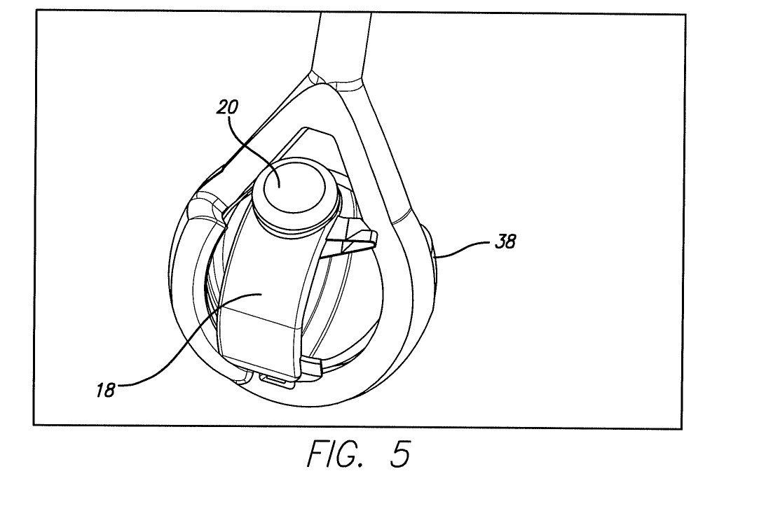

Referring to FIG. 5 is a perspective view of the test device in implant holder 38. The test device suspension system moves the test device implant holder in the water-jacketed cell 22. Implant Suspension Fixture and Silicone Eye. The suspension system is inserted into the heated PBS solution and mimics the saccadic movement of a human eye over an equivalent of five years. The suspension fixture secures a silicone eye in which the implant is mounted on. It has several features that are designed to maximize performance including: Silicone eye flange and internal groove; Fins; Gaps at top and bottom; and Removable accelerometer pad. The suspension fixture has an internal groove that matches the external flange on the diameter of the silicone eye. This flange and groove serve multiple purposes. Firstly, the combination securely locks the silicone eye in the suspension fixture and prevents the silicone eye from twisting inside the fixture during testing. Secondly, it locks the PBS, inside the silicone eye.

Heated water is pumped through the glass water-jacket cell 22 on the bottom. The heated glass jacket heats the interior flask and the contents inside the flask (i.e. the implant 20). The water-jacketed cell 22 lid contains a bearing and hole for the stirrer that we can re-drill elsewhere. Twoside openings can be used for the thermocouple and for the sensing electrodes.

The water-jacketed cell 22 keeps the interior liquid separate from exterior liquid. The Stabilizing Cap Fixture (SCF) or motor holder 32 will provide a rigid structure to mount the motor and primary communication coil;

The SCF is a system that connects directly with the solid cap bought from Bellco Glass. This fixture consists of multiple pieces including motor mount and adjustable arm that mechanically supports the primary coil 16. The motor mount is screwed directly into the solid cap. During initial assembly, the motor should be aligned with the holes before the handle is screwed into the solid cap. This ensures that the motor is aligned with the shaft hole. The adjustable arm that holds the primary coil 16 adjusts both angularly and vertically. This ensures that the primary coil 16 location can be adjusted to reach the 10-20 mm distance and the proper angular orientation from the implant to ensure good communication.

Since the SCF fixes to the cap and vessel, the SCF will be immobile in relation to the rest of the system. If there is an earthquake, or if the table is knocked, or for some other reason the vessel is moved from its place on the table, the structure will move with it, and the testing apparatus will continue to function normally.

The SCF Structure attaches directly to vessel forming the water jacketed cell 22. This ensures that the motor shaft is aligned horizontally with the vessel. The IDLT components use only a small footprint.

The sensing system 10 is controlled by a field programmable gate array (FPGA). The FPGA and surrounding board are configured with a program called the Embedded Development Kit (EDK). This program allows for onboard FPGA resources to be transformed into Intellectual Property (IP) cores. These IP cores allow for the FPGA to do useful and specific work with significantly less programming for the user. IP cores also allow for the FPGA resources to communicate to other board peripherals in order to process signals. 16 MB Intel StrataFlash parallel NOR Flash PROM; Stores multiple FPGA configurations and dynamically switches between the different configurations with the Spartan-3E's MultiBoot feature; Stores and executes MicroBlaze processor code directly from the StrataFlash device; Stores MicroBlaze code on the StrataFlash device and shadows the code into DDR memory before executing the code; Stores non-volatile data from FPGA on the StrataFlash SPI Operation; A/D and DAC connection to FPGA; Expansion bay peripherals connection to FPGA; SPI Serial Flash (16 Mbits); Simple non-volatile storage; Useful for storage of codes, serial numbers, IP addresses, etc.; Similar to StrataFlash: Allows for storage of MicroBlaze code that can be shadowed into DDR SDRAM; 10/100 Ethernet Physical Layer Interface Useful for networking multiple test devices for control by one PC; Along with the specified IP core (hardware specification for FPGA), allows for simple control by MicroBlaze

Dual RS-232 Ports;. Dual ports allow for connection to the serial-based heat pump as well as connection to a PC for debugging and development. This board contains all of the features that are required for correct interfacing to all of our chosen sensors as well as to the PC. The addition of the non-volatile flash memory, multiple dynamic boot options, and shadowing to the DDR SDRAM were great pluses in our analysis of other boards.

Silicon structures are attached to the substrate in a few points called anchors, freely move in the direction of the sensed acceleration. The proof mass displaces from its nominal position, causing an imbalance. This imbalance applies a voltage pulse to a low noise capacitive amplifier which converts the pulse into an analog voltage. With three integrated ADC, one for each axis, the signal is translated and produced into a digital bit stream. This specific model is linear, but by using the functions below we are able to calculate its angular parameters.

A three Axis accelerometer makes it easy to work with no matter how we choose to orient it. Adjustable bandwidth eliminates interference from irrelevant frequency areas and direction detection which signals threshold crossing along any of 3 axes.

Using a reservoir tank to Heat and Refrigerate fluids, the heat pump 24 provides a closed/Open loop connection to an external apparatus maintaining adequate flow at all times. This provides precise temperature control of fluids; A Rapid Response Heater heat up quickly and add stability when process or ambient conditions change rapidly. The Variable Speed Duplex Pump includes an RS 232 Interface for control.

Upon boot, the microcontroller will run through an initialization sequence and a series of self-diagnostic tests to ensure that the microcontroller is operational. Sensor and Motor configurations follows, which requires the configuration the RS-232 and the Serial Peripheral Interface.

Accordingly, what has been shown is an improved test apparatus and method of implantable devices. While the invention has been described by means of specific embodiments and applications thereof, it is understood that numerous modifications and variations could be made thereto by those skilled in the art without departing from the spirit and scope of the invention. It is therefore to be understood that within the scope of the claims, the invention may be practiced otherwise than as specifically described herein.

We claim:

1. An apparatus for testing long term reliability of an implantable device comprising:
a vessel of saline;
mean for controlling temperature of the saline;
means for supporting and repetitively moving a test device within the vessel;
means for providing a stimulation signal to the test device; and
means for testing the electrical integrity of the test device.

2. The apparatus for testing long term reliability of an implantable device according to claim 1, wherein the means for testing electrical integrity is a voltmeter.

3. The apparatus for testing long term reliability of an implantable device according to claim 1, wherein the means for testing electrical integrity is a anuneter.

4. The apparatus for testing long term reliability of an implantable device according to claim 1, wherein the means for testing electrical integrity is a ohmeter.

5. The apparatus for testing long term reliability of an implantable device according to claim 1, wherein the means for testing electrical integrity is a electrometer.

6. The apparatus for testing long term reliability of an implantable device according to claim 1, wherein the means for testing electrical integrity is a potentiostat.

7. The apparatus for testing long term reliability of an implantable device according to claim 1, wherein the means for providing a stimulation signal provides an alternating current.

8. The apparatus for testing long term reliability of an implantable device according to claim 1, wherein the means for providing a stimulation signal provides a alternating voltage.

9. The apparatus for testing long term reliability of an implantable device according to claim 1, further comprising a computer controlling the means for providing a stimulation signal.

10. The apparatus for testing long term reliability of an implantable device according to claim 9, wherein the computer records data from the means for testing the electrical integrity of the test device.

11. The apparatus for testing long term reliability of an implantable device according to claim 1, wherein the means for supporting and repetitively moving a test device within the vessel is a stepper motor.

12. The apparatus for testing long term reliability of an implantable device according to claim 1, wherein the means for supporting and repetitively moving a test device within the vessel is a three axis accelerometer.

13. The apparatus for testing long term reliability of an implantable device according to claim 1, wherein the means for supporting and repetitively moving a test device within the vessel mimics the movement of any eye.

14. The apparatus for testing long term reliability of an implantable device according to claim 1, wherein the mean for controlling temperature of the saline is a water jacket separated from the saline.

15. The apparatus for testing long term reliability of an implantable device according to claim 1, wherein the saline is phosphate buffered saline.

16. An method of testing the long term reliability of an implantable device comprising:
providing a vessel of saline;
controlling the temperature of the saline;
supporting and repetitively moving a test device within the vessel;
providing a stimulation signal to the test device; and
testing the electrical integrity of the test device.

17. The method according to claim 16, wherein testing is testing with a voltmeter.

18. The method according to claim 16, wherein moving a test device is rotating the test device.

19. The method according to claim 16, wherein moving a test device is moving the test device in a three axis motion.

20. The method according to claim 16, wherein controlling the temperature of the saline is controlling the temperature of the saline with a water jacket.
